(19) United States
(12) Patent Application Publication
(10) Pub. No.: US 2009/0239882 A1
(43) Pub. Date: Sep. 24, 2009

(54) Thiazolopyramidine Compounds for the Modulation of Chemokine Receptor Activity

(75) Inventors: Premji Meghani, Leicestershire (GB); David Ranulf Cheshire, Leicestershire (GB); Cherylin Francis Preston, Devon (GB); Jeffrey Paul Stonehouse, Leicestershire (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/721,583

(22) PCT Filed: Dec. 14, 2005
(86) PCT No.: PCT/GB05/04825

§ 371 Date: Jun. 13, 2007

(30) Foreign Application Priority Data
Dec. 17, 2004 (GB) 0427698.6
Feb. 8, 2005 (GB) 0502542.4

(51) Int. Cl. A61K 31/519; C07D 513/04; A61P 35/00

(52) U.S. Cl. 514/260.1; 544/255

(57) ABSTRACT

A compound of formula (I), or a pharmaceutically acceptable salt, or solvate thereof; and pharmaceutical compositions comprising these, all for use in the treatment of chemokine mediated diseases and disorders.

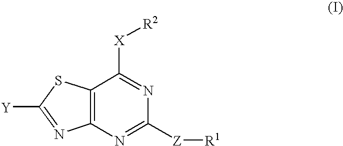

The present invention relates to certain heterocyclic compounds, processes and intermediates used in their preparation, pharmaceutical compositions containing them and their use in therapy.

Chemokines play an important role in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved cysteine motif. At the present time, the chemokine superfamily comprises four groups exhibiting characteristic structural motifs, the C—X—C, C-C and C—X₃—C and XC families. The C—X—C and C-C families have sequence similarity and are distinguished from one another on the basis of a single amino acid insertion between the NH-proximal pair of cysteine residues. The C—X₃—C family is distinguished from the other two families on the basis of having a triple amino acid insertion between the NH-proximal pair of cysteine residues. In contrast, members of the XC family lack one of the first two cysteine residues.

The C—X—C chemokines include several potent chemoattractants and activators of neutrophils such as interleukin-8 (IL-8) and neutrophil-activating peptide 2 (NAP-2).

The C-C chemokines include potent chemoattractants of monocytes and lymphocytes but not neutrophils. Examples include human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MEP-1α and MIP-1β).

The C—X₃—C chemokine (also known as fractalkine) is a potent chemoattractant and activator of microglia in the central nervous system (CNS) as well as of monocytes, T cells, NK cells and mast cells.

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 (for the C—X—C family) and CX₃CR¹ for the C—X₃—C family. These receptors represent good targets for drug development since agents which modulate these receptors would be useful in the treatment of disorders and diseases such as those mentioned above. In accordance with the present invention, there is therefore provided a compound of general formula (I)

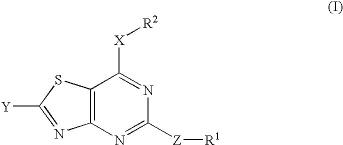

wherein

R¹ is a group selected from C₃₋₇-carbocyclyl, C₁₋₈alkyl, C₂₋₆alkenyl and C₂₋₆alkynyl; wherein the group is optionally substituted by 1, 2 or 3 substituents independently selected from fluoro, nitrile, —OR⁴, —NR⁵R⁶, —CONR⁵R⁶, —COOR⁷, —N⁸COR⁹, —SR¹⁰, —SO₂R¹⁰, —SO₂NR⁵R⁶, —NR⁸SO₂R⁹, phenyl or heteroaryl; wherein phenyl and heteroaryl are optionally substituted by 1, 2 or 3 substituents independently selected from halo, cyano, nitro, —OR⁴, —NR⁵R⁶, —CONR⁵R⁶, —COOR⁷, —NR⁸COR⁹, —SR¹⁰, —SO₂R¹⁰, —SO₂NR⁵R⁶, —NR⁸SO₂R⁹, alkyl and trifluoromethyl;

X is —CH₂—, a bond, oxygen, sulphur, sulphoxide, or sulphone;

Z is —CH₂—, a bond, oxygen, sulphur, sulphoxide, sulphone or —NR⁵;

R² is C₃₋₇-carbocyclyl, optionally substituted by 1, 2 or 3 substituents independently selected from fluoro, —OR⁴, —NR⁵R⁶—CONR⁵R⁶, —COOR⁷, —NR⁸COR⁹, —SR¹⁰, —SO₂R¹⁰, —SO₂NR⁵R⁶, —NR⁸SO₂R⁹;

or R² is a 3-8 membered ring optionally containing 1, 2 or 3 atoms selected from O, S, —NR⁸ and whereby the ring is optionally substituted by 1, 2 or 3 substituents independently selected from C₁₋₃alkyl, fluoro, —OR⁴, —NR⁵R⁶—CONR⁵R⁶, —COOR⁷, —NR⁸COR⁹, —SR¹⁰, —SO₂R¹⁰, —SO₂NR⁵R⁶, —NR⁸SO₂R⁹;
or R² is phenyl or heteroaryl, each of which is optionally substituted by 1, 2 or 3 substituents independently selected from halo, cyano, nitro, —OR⁴, —NR⁵R⁶, —CONR⁵R⁶, —NR⁸COR⁹, —SO₂NR⁵R⁶, —NR⁸SO₂R⁹, C₁₋₆alkyl and trifluoromethyl;
or R² is a group selected from C₁₋₈alkyl, C₂₋₆alkenyl or C₂₋₆alkynyl wherein the group is substituted by 1, 2 or 3 substituents independently selected from hydroxy, amino, C₁₋₆alkoxy, C₁₋₆alkylamino, di(C₁₋₆alkyl)amino, N—(C₁₋₆alkyl)-N-(phenyl)amino, N—C₁₋₆alkylcarbamoyl, N,N-di(C₁₋₆alkyl)carbamoyl, N—(C₁₋₆alkyl)-N-(phenyl)carbamoyl, carboxy, phenoxycarbonyl, —NR⁸COR⁹, —SO₂R¹⁰, —SO₂NR⁵R⁶, —NR⁸SO₂R⁹ and —CONR⁵R⁶;

Y is selected from hydrogen, hydroxyl, halo, —NR³R⁴, and —NR⁸SO₂R⁹;

R³ and R⁴ each independently represent a hydrogen atom, or a 4-piperidinyl group, or R³ and R⁴ each independently represent a C₃-C₆ cycloalkyl or C₁-C₈ alkyl group, which groups may be optionally substituted by 1, 2 or 3 substituent groups independently selected from halo, —NR⁵R⁶, —CONR⁵R⁶, —OR⁷, —COOR⁷, —NR⁸COR⁹, —SR¹⁰, —SO₂R¹⁰, —SO₂NR⁵R⁶, —NR⁸SO₂R⁹, morpholinyl, C₁-C₄ alkyl, C₃-C₆ cycloalkyl, tetrahydrofuranyl and aryl, wherein an aryl group may be optionally substituted by 1, 2 or 3 substituents independently selected from halo, cyano, nitro, —NR⁵R⁶, —CONR⁵R⁶, —OR⁷, —NR⁸COR⁹, —SO₂NR⁵R⁶, —NR⁸SO₂R⁹, C₁-C₆ alkyl and trifluoromethyl, or R³ and R⁴ together with the nitrogen atom to which they are attached form a 4- to 7-membered saturated heterocyclic ring system, which ring system may be optionally substituted by one or more substituent groups independently selected from

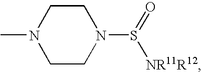

—NR⁵R⁶, —CONR⁵R⁶, —OR⁷, —COOR¹⁰, —NR⁸COR⁹, and C₁-C₆ alkyl optionally substituted by 1, 2 or 3 substituents independently selected from halogen atoms and —NR¹¹R¹² and —OR⁷ groups;

R⁵ and R⁶ are independently selected from hydrogen or a group selected from C₁₋₆alkyl and phenyl wherein the group is optionally substituted by 1, 2 or 3 substituents independently selected from halo, phenyl, —OR¹⁴, —NR¹⁵R¹⁶, —COOR¹⁴, —CONR¹⁵R¹⁶, —NR¹⁵COR¹⁶, —SO₂R¹⁰, —SONR¹⁵R¹⁶ and NR—SO₂R¹⁶

R⁷ and R⁹ each independently represent a hydrogen atom or a C₁-C₆, particularly C₁-C₄, alkyl (e.g. methyl, ethyl, propyl, butyl, pentyl or hexyl) or phenyl group, each of which may be optionally substituted by one or more (e.g. one, two, three or four) substituent groups independently selected from halogen atoms (e.g. fluorine, chlorine, bromine or iodine), phenyl, —OR¹⁷ and —NR¹⁵R¹⁶; and

each of R⁸, R¹⁰, R¹¹, R¹², R¹⁵, R¹⁶ and R¹⁷ independently represents a hydrogen atom or a C₁-C₆, particularly C₁-C₄, alkyl (e.g. methyl, ethyl, propyl, butyl, pentyl or hexyl) or phenyl group
or a pharmaceutically acceptable salt or solvate thereof.

Convenient values of R¹, R², X, Z and Y are as follows. Such values may be used independently where appropriate with any of the definitions, claims or embodiments defined hereinbefore or hereinafter.

R¹ is C₁₋₈alkyl optionally substituted by 1, 2 or 3 substituents independently selected from nitrile, phenyl or heteroaryl, wherein phenyl and heteroaryl are optionally substituted by 1, 2 or 3 substituents independently selected from halo, cyano, —OR⁴, —SR¹⁰, C₁₋₆alkyl and trifluoromethyl wherein R⁴ and R¹⁰ are as defined in formula (I); or

R¹ is C₁₋₄alkyl optionally substituted by 1, 2 or 3 substituents independently selected from phenyl or heteroaryl, wherein phenyl and heteroaryl are optionally substituted by 1, 2 or 3 substituents independently selected from halo, cyano, —OR⁴, and trifluoromethyl wherein R⁴ is as defined in formula (I); or

R¹ is C₁₋₂alkyl substituted by phenyl optionally substituted by 1, 2, or 3 substituents independently selected from fluoro, chloro, bromo, methoxy, methyl and trifluoromethyl; or

R¹ is C₁₋₂alkyl substituted by phenyl optionally substituted by 1, 2, or 3 substituents independently selected from fluoro, chloro, bromo, methoxy, methyl and trifluoromethyl.

R² is C₁₋₈alkyl substituted by 1, 2 or 3 substituents independently selected from hydroxy, amino, C₁₋₆alkoxy, C₁₋₆alkylamino, carboxy, —NR⁸COR⁹, —SO₂R¹⁰, —SO₂NR⁵R⁶, —NR⁸SO₂R⁹ and —CONR⁵R⁶, wherein R⁵, R⁶, R⁸, R⁹ and R¹⁰ are as defined in formula (I); or

R² is C₁₋₈alkyl substituted with 1 or 2 hydroxy groups, carboxy, —NR⁸COR⁹ or —CONR⁵R⁶; wherein R⁵, R⁶, R⁸ and R⁹ are as defined in formula (I); or

R² is C₁₋₄alkyl substituted with 1 or 2 hydroxy groups, carboxy, —NHCOC₁₋₄alkyl or —CONR⁵R⁶ wherein R⁵ and R⁶ are either hydrogen or C₁₋₄alkyl; or

R² is C₁₋₄alkyl substituted with 1 or 2 hydroxy groups.

It will be appreciated that when R² is a 3-8 membered ring optionally containing 1, 2 or 3 atoms selected from O, S, —NR⁸ then such ring is linked via a carbon ring atom (i.e. it is not linked to X via the optional heteroatom).

X is a bond, —CH₂—, oxygen, or sulphur; or X is a bond or oxygen

In a further aspect of the present invention there is provided a compound of formula (1) as depicted above in which Z is a bond, —CH₂—, oxygen, sulphur or NR⁵.

In another aspect of the invention Z is a bond, —CH₂—, oxygen or sulphur.

In another aspect of the invention Z is a bond, —CH₂— or sulphur

In another aspect of the invention Z is a bond or sulphur

In a further aspect of the present invention there is provided a compound of formula (I) as depicted above wherein Y is a hydrogen atom, or a group hydroxyl, —NR³R⁴ or —NR⁸SO₂R⁹ wherein R³, R⁴, R⁸ and R⁹ are as defined in formula (I).

In another aspect of the invention Y is hydroxyl, —NR³R⁴ or —NR⁸SO₂R⁹ wherein R³, R⁴, R⁸ and R⁹ are as defined in formula (I).

In another aspect of the invention Y is hydroxyl, —NR³R⁴ or —NR⁸SO₂R⁹ wherein R³, R⁴, R⁸ are either hydrogen or C₁₋₄alkyl and R⁹ is either C₁₋₄alkyl or trifloromethyl.

In another aspect of the invention Y is hydroxyl, —NH₂ or —NHSO₂Me.

Particular compounds of the invention include:
3-[[2-amino-5-[[(2-fluorophenyl)methyl]thio]thiazolo[4,5-d]pyrimidin-7-yl]oxy]-propanoic acid,
3-[[2-amino-5-[[(2-fluorophenyl)methyl]thio]thiazolo[4,5-d]pyrimidin-7-yl]oxy]-propanamide,
N-[2-[[2-amino-5-[[(2-fluorophenyl)methyl]thio]thiazolo[4,5-d]pyrimidin-7-yl]oxy]ethyl]-acetamide,
1-propanol, 2-[[2-amino-5-[[(3-chloro-4-methoxyphenyl)methyl]thio]thiazolo[4,5-d]pyrimidin-7-yl]oxy]-, (2R)—,
(2R)-2-({2-amino-5-[(2,3-difluorobenzyl)thio][1,3]thiazolo[4,5-d]pyrimidin-7-yl}oxy)propan-1-ol, and
(2S)-2-({2-amino-5-[(2,3-difluorobenzyl)thio][1,3]thiazolo[4,5-d]pyrimidin-7-yl}oxy)propan-1-ol
5-[(2,3-difluorobenzyl)thio]-7-[(1R)-2-hydroxy-1-methylethoxy][1,3]thiazolo[4,5-d]pyrimidin-2(3H)-one

Each of the above mentioned compounds and the pharmaceutically acceptable salts, solvates or in vivo hydrolysable esters thereof, taken individually is a preferred aspect of the invention.

According to the invention there is also provided a process for the preparation of a compound of formula (I) which comprises:

When X represents —O— or —S—, and Z, R¹ and Y are as defined in formula (I), with the proviso that Y is not hydroxyl, reacting a compound of general formula (II)

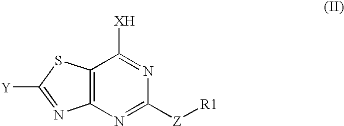

with a suitable alkylhalide (R²-L) wherein R² is as defined in formula (I) and L is a leaving group such as halogen, alkyl- or aryl-sulphonate or activated alcohol under standard Mitsunobu reaction conditions (Synthesis 1, 1981) using trialkyl- or triaryl-phosphine and dialkylazidodicarboxylate in the presence of a suitable base and solvent. Examples of suitable bases include trialkylamines, such as triethylamine or N,N-diisopropylemylamine, pyridine or 4-dimethylaminopyridine or alkali metal hydroxides such as Li, Na, or K, or metal carbonates such as Li, Na, K or Cs, or metal acetates such as Li, Na, K or Cs, or metal alkoxides such as Li, Na, K-tert-butoxide. Preferably triethylamine or N,N-diisopropylemylamine is used. Suitable trialkyl- or triaryl-phosphines include tri-n-butylphosphine or triphenylphosphine. Suitable dialkylazidodicarboxylates include diethylazidodicarboxylate or diiosopropylazidodicarboxylate. Suitable solvents include dichloromethane, pyridine, N,N-dimethylamides, 1-methyl-2-pyrolidone, and ethers such as tetrahydrofuran, 1,4-dioxane, glyme and diglyme. Preferably N,N-dimethylformamide or tetrahydrofuran is used. The temperature of the reaction can be performed between 0° C. and 120° C.

Compounds of Formula (II) may be prepared as described in our published PCT patent application WO0009511 (AstraZeneca).

According to the invention there is also provided a process for the preparation of a compound of formula (I), wherein Y is hydroxyl, or a pharmaceutically acceptable salt or solvate thereof, which process comprises reacting a compound of general formula (III); wherein L is a leaving group and PG is a suitable protecting group

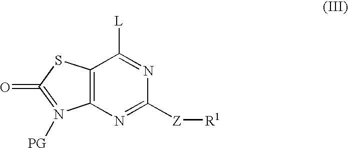

with suitable nucleophiles (e.g. HX—R², (Metal)X—R², Mg(Halogen)X—R²) wherein R² is as defined in formula (I) in the presence or absence of a suitable base and solvent. Preferably L is a halogen. Examples of suitable protecting groups include tetrahydropyranyl, trimethylsilylethoxymethyl, phenyloxymethyl, methyloxymethyl, benzyloxymethyl, phenylethylsulfone and propionitrile. Preferably tetrahydropyranyl is used. Examples of suitable bases include trialkylamines, such as triethylamine or N,N-diisopropylethylamine, pyridine or 4-dimethylaminopyridine or alkali metal hydrides such as Li, Na, or K or alkali metal hydroxides such as Li, Na, or K, or metal carbonates such as Li, Na, K or Cs, or metal acetates such as Li, Na, K or Cs, or metal alkoxides such as Li, Na, K tert-butoxide. Preferably sodium hydride is used. Suitable solvents include dichloromethane, pyridine, N,N-dimethylamides, 1-methyl-2-pyrrolidinone, and ethers such as tetrahydrofuran, 1,4-dioxane, glyme and diglyme. Preferably N,N-dimethylformamide or tetrahydrofuran is used. Examples of suitable metals are Li, Na, K, Zn, Ce, Cu, Sn, and Pd. The temperature of the reaction can be performed between 0° C. and 120° C.

Compounds of formula (I), wherein X and/or Z are sulphoxide or sulphone and R¹ and R² are as defined hereinbefore, may be prepared by further reaction of compounds of formula (I), wherein X and/or Z are sulphur, with a suitable oxidising reagent. Examples of suitable oxidising reagents include, hydrogen peroxide, sodium periodate, periodic acid, peroxy acids such as metachloroperbenzoic acid and peracetic acid and oxone.

Compounds of Formula (III), wherein Y is hydroxyl and R¹, Z, L and PG are as described hereinbefore, may be prepared from compounds of formula (IV) wherein R¹ and Z are as defined in formula (I) and L is a halogen by reaction with a suitable protecting group reagent under appropriate reaction conditions as is fully described in ‘Protective Groups in Organic Chemistry’, edited by J. W. F. McOmie, Plenum Press (1973), and ‘Protective Groups in Organic Synthesis’, 2^{nd} Edition, T. W. Greene & P. G. M. Wuts, Wiley Interscience (1991).

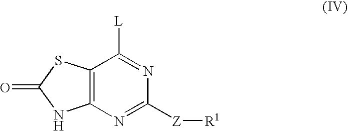

Compounds of Formula (IV), wherein Y is hydroxyl and R¹, Z, and L are as defined hereinbefore may be prepared from compounds of formula (V) wherein R¹ and Z are as defined in formula (I)

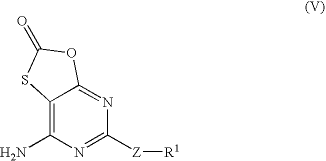

by treatment with phosphorous oxychloride in the presence of a phase transfer reagent and suitable solvent. Examples of phase transfer reagents include tetraalkylammonium halides and tetraalkylphosphonium halides. Preferably benzyltrimethylammonium chloride is used. Suitable solvents include acetonitrile, toluene, xylene, N,N-dimethylaniline, N,N-diethylaniline, 1,2-dimethoxyethane and ethers such as tetrahydrofuran, 1,4-dioxane, glyme and diglyme. Preferably a mixture of acetonitrile and N,N-diethylaniline is used. The reaction can be performed between temperatures of 30° C. and 120° C.

Compounds of formula (V), wherein Y is hydroxyl and R¹ and Z are as defined hereinbefore, may be prepared from compounds of formula (VI), wherein R¹ and Z are as defined in formula (I)

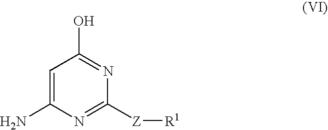

by treatment with a halocarbonylsulfenyl halide in the presence of suitable solvent. Covenient halogen atoms are independently selected from chlorine and bromine. Preferably chlorine as the halogen atom is used, and chlorocarbonylsulfenyl chloride is therefore the preferred reagent. Examples of suitable solvents include dichloromethane, and ethers such as tetrahydrofuran, 1,4-dioxane, 1,2-dimethoxyethane, glyme, diglyme and diethylether. Tetrahydrofuran and diethylether are preferred. The reaction can be performed between temperatures of 0° C. and 50° C.

Compounds of formula (VI), wherein Y is hydroxyl and Z represents —O— or —S— and R¹ is as defined hereinbefore, may be prepared from a compound of formula (VII) by treatment with an alkyl halide (R¹L), wherein R¹ is as defined in formula (I) and L is a leaving group such as halogen or alkyl- or aryl-sulfonate

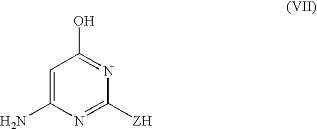

in the presence of suitable base and solvent. Examples of suitable base include trialkylamines, such as triethylamine or N,N-diisopropylemylamine, pyridine or 4-dimethylaminopyridine or alkali metal hydroxides such as Li, Na or K, or metal carbonates such as Li, Na, K or Cs, or metal alkoxides such as Li, Na, K or Cs, or metal acetates such as Li, Na, K, or Cs or metal alkoxides such as Li, Na, K tert-butoxide. Preferably sodium acetate or sodium hydroxide is used. Examples of suitable solvent include acetonitrile, pyridine, N,N-dimethylamides, 2-methyl-1-pyrollidone, and ethers such as tetrahydrofuran, 1,4-dioxane, glyme and diglyme. Preferably N,N-dimethylformamide or acetonitrile is used. The reaction is performed at temperatures between 0° C. and 120° C.

The compounds of formula (I) have activity as pharmaceuticals, in particular as modulators of chemokine receptor (especially CXCR2) activity, and may be used in the treatment (therapeutic or prophylactic) of conditions/diseases in human and non-human animals which are exacerbated or caused by excessive or unregulated production of chemokines.

Certain compounds of formula (I) are capable of existing in stereoisomeric forms. It will be understood that the invention encompasses all geometric and optical isomers of the compounds of formula (I) and mixtures thereof including racemates.

The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

Within the present invention it is to be understood that a compound of formula (I) or a salt, solvate or in vivo hydrolysable ester thereof may exhibit the phenomenon of tautomerism and that the formulae drawings within this specification can represent only one of the possible tautomeric forms. It is to be understood that the invention encompasses any tautomeric form and mixtures thereof and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein.

It is also to be understood that certain compounds of formula (I) and salts thereof can exist in solvated as well as unsolvated forms such as, for example, hydrated forms. It is to be understood that the invention encompasses all such solvated forms.

The present invention relates to the compounds of formula (I) as hereinbefore defined as well as to the salts thereof. Salts for use in pharmaceutical compositions will be pharmaceutically acceptable salts, but other salts may be useful in the production of the compounds of formula (I) and their pharmaceutically acceptable salts. Pharmaceutically acceptable salts of the invention may, for example, include acid addition salts of the compounds of formula (I) as hereinbefore defined which are sufficiently basic to form such salts. Such acid addition salts include for example salts with inorganic or organic acids affording pharmaceutically acceptable anions such as with hydrogen halides (especially hydrochloric or hydrobromic acid of which hydrochloric acid is particularly preferred) or with sulphuric or phosphoric acid, or with trifluoroacetic, citric or maleic acid. Suitable salts include hydrochlorides, hydrobromides, phosphates, sulphates, hydrogen sulphates, alkylsulphonates, arylsulphonates, acetates, benzoates, citrates, maleates, fumarates, succinates, lactates, tartrates, oxalates, methanesulphonates or p-toluenesulphonates. Pharmaceutically acceptable salts of the invention may also include basic addition salts of the compounds of formula (I) as hereinbefore defined which are sufficiently acidic to form such salts. Such salts may be formed with an inorganic or organic base which affords a pharmaceutically acceptable cation. Such salts with inorganic or organic bases include for example an alkali metal salt, such as a lithium, sodium or potassium salt, an alkaline earth metal salt such as a calcium or magnesium salt, an ammonium salt or an organic amine salt, for example a salt with methylamine, dimethylamine, trimethylamine, triethylamine, piperidine, morpholine or tris-(2-hydroxyethyl)amine. Other basic addition salts include aluminium, zinc, benzathine, chloroprocaine, choline, diethanolamine, ethanolamine, ethyldiamine, meglumine, tromethamine or procaine.

The present invention further relates to an in vivo hydrolysable ester of a compound of formula (I). An in vivo hydrolysable ester of a compound of formula (I) which contains carboxy or hydroxy group is, for example a pharmaceutically acceptable ester which is cleaved in the human or animal body to produce the parent acid or alcohol. Such esters can be identified by administering, for example, intravenously to a test animal, the compound under test and subsequently examining the test animal's body fluid.

Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

Suitable pharmaceutically-acceptable esters for hydroxy include inorganic esters such as phosphate esters (including phosphoramidic cyclic esters) and α-acyloxyalkyl ethers and related compounds which as a result of the in vivo hydrolysis of the ester breakdown to give the parent hydroxy group/s. Examples of α-acyloxyalkyl ethers include acetoxymethoxy and 2,2-dimethylpropionyloxymethoxy. A selection of in-vivo hydrolysable ester forming groups for hydroxy include C₁₋₁₀alkanoyl, for example acetyl; benzoyl; phenylacetyl; substituted benzoyl and phenylacetyl, C₁₋₁₀alkoxycarbonyl (to give alkyl carbonate esters), for example ethoxycarbonyl; di-(C₁₋₄)alkylcarbamoyl and N-(di-(C₁₋₄)alkylaminoethyl)-N-(C₁₋₄)alkylcarbamoyl (to give carbamates); di-(C₁₋₄)alkylaminoacetyl and carboxyacetyl. Examples of ring substituents on phenylacetyl and benzoyl include aminomethyl, (C₁₋₄)alkylaminomethyl and di-((C₁₋₄)alkyl)aminomethyl, and morpholino or piperazino linked from a ring nitrogen atom via a methylene linking group to the 3- or 4-position of the benzoyl ring. Other interesting in-vivo hydrolysable esters include, for example, R^{A}C(O)O(C₁₋₆)alkyl-CO—, wherein R^{A} is for example, benzyloxy-(C₁₋₄)alkyl, or phenyl). Suitable substituents on a phenyl group in such esters include, for example, 4-(C₁₋₄)piperazino-(C₁₋₄)alkyl, piperazino-(C₁₋₄)alkyl and morpholino-(C₁₋₄)alkyl.

The compounds of formula (1) above may be converted to a pharmaceutically acceptable salt, solvate or in vivo hydrolysable ester thereof, as discussed above. The salt is preferably a basic addition salt.

In the context of the present specification, unless otherwise indicated, an alkyl or alkenyl group or an alkyl or alkenyl moiety in a substituent group may be linear or branched. Where a substituent in an alkenyl group is a phenoxy group, the phenoxy group is not attached to an unsaturated carbon atom A carbocyclic group is a saturated hydrocarbyl group that may be monocyclic or polycyclic (e.g. bicyclic). Similarly, a saturated heterocyclic ring system may be monocyclic or polycyclic (e.g. bicyclic).

In this specification the term “alkyl” includes both straight-chain and branched-chain alkyl groups. However references to individual alkyl groups such as “propyl” are specific for the straight chain version only and references to individual branched-chain alkyl groups such as f-butyl are specific for the branched chain version only. For example, “C₁₋₃alkyl” includes methyl, ethyl, propyl and isopropyl and examples of “C₁₋₆alkyl” include the examples of “C₁₋₃alkyl” and additionally t-butyl, pentyl, 2,3-dimethylpropyl, 3-methylbutyl and hexyl. Examples of “C₁₋₈alkyl” include the examples of “C₁₋₆alkyl” and additionally heptyl, 2,3-dimethylpentyl, 1-propylbutyl and octyl. An analogous convention applies to other terms, for example “C₂₋₆alkenyl” includes vinyl, allyl, 1-propenyl, 2-butenyl, 3-butenyl, 3-methylbut-1-enyl, 1-pentenyl and 4-hexenyl and examples of “C₂₋₆alkynyl” includes ethynyl, 1-propynyl, 3-butynyl, 2-pentynyl and 1-methylpent-2-ynyl.

“C₃₋₇carbocyclyl” is a saturated, partially saturated or unsaturated, monocyclic ring containing 3 to 7 carbon ring atoms wherein a —CH₂— group can optionally be replaced by a —C(O)—. Suitable examples of “carbocyclyl” are cyclopropyl, cyclopentyl, cyclobutyl, cyclohexyl, cyclohexenyl, 4-oxocyclohex-1-yl and 3-oxocyclohept-5-en-1-yl.

The term “halo” refers to fluoro, chloro, bromo and iodo.

Examples of “C₁₋₆alkoxy” include methoxy, ethoxy, propoxy, isopropoxy, butyloxy, pentyloxy, 1-ethylpropoxy and hexyloxy. Examples of “C₁₋₆alkylamino” include methylamino, ethylamino, propylamino, butylamino and 2-methylpropylmino. Examples of “di(C₁₋₆alkyl)amino” include dimethylamino, N-methyl-N-ethylamino, diethylamino, N-propyl-N-3-methylbutylamino. Examples of “N-(C₁₋₆alkyl)-N-(phenyl)amino” include N-methyl-N-phenylamino, N-propyl-N-phenylamino and N-(2-methylbutyl)-N-phenylamino. Examples of “N—(C₁₋₆alkyl)carbamoyl” are N-methylcarbamoyl, N-ethylcarbamoyl and N-(2-ethylbutylcarbamoyl. Examples of “N-(C₁₋₆alkyl)-N-(phenyl)carbamoyl” include N-methyl-N-phenylcarbamoyl, N-butyl-N-phenylcarbamoyl and N-(3-methylpentyl)-N-(phenyl)carbamoyl. Examples of “N,N-di(C₁₋₆alkyl)carbamoyl” include N,N-dimethylcarbamoyl, N-methyl-N-ethylcarbamoyl and N-propyl-N-(2-methylbutyl)carbamoyl. Examples of “C₁₋₆alkylthio” include methylthio, ethylthio, propylthio, butylthio and 2-methylbutylthio.

“Heteroaryl” is a monocyclic or bicyclic aryl ring containing 5 to 10 ring atoms of which 1, 2, 3 or 4 ring atoms are chosen from nitrogen, sulphur or oxygen. Examples of heteroaryl include pyrrolyl, furanyl, thienyl, thiazolyl, isothiazolyl, oxazolyl, isoxazolyl, imidazolyl, pyrazolyl, triazolyl, tetrazolyl, pyridyl, pyrimidinyl, pyrazinyl, pyridazinyl, triazinyl, benzfuranyl, benzthieno, indolyl, benzimidazolyl, benzoxazolyl, benzthiazolyl, indazolyl, benzisoxazolyl, benzisothiazolyl, benztriazolyl, quinolinyl, isoquinolinyl and naphthiridinyl. Conveniently heteroaryl is selected from imidazolyl, pyrazolyl, thiazolyl, isoxazolyl, furanyl, thienyl, isoxazolyl, or indazolyl. Fully saturated heterocyclic rings include examples such as oxetanyl, azetidinyl, pyrrolidinyl, tetrahydrofuranyl, tetrahydropyranyl, piperidinyl, piperazinyl, morpholinyl, homopiperidinyl and homopiperazinyl and tetrahydropyridinyl.

Examples of “a 3-8 membered ring optionally containing 1, 2 or 3 atoms selected from O, S and NR⁸” include saturated rings such as oxetanyl, azetidinyl, benzodiazolyl, pyrrolidinyl, tetrahydrofuranyl, tetrahydrothiophenyl, tetrahydropyranyl, piperidinyl, piperazinyl, morpholinyl, homopiperidinyl and homopiperazinyl tetrahydrodioxanyl. Examples of “a 4- to 7-membered saturated heterocyclic ring system” include azetidinyl, pyrrolidinyl, piperidinyl, piperazinyl, homopiperazinyl and morpholinyl.

Where optional substituents are chosen from “1, 2 or 3” groups it is to be understood that this definition includes all substituents being chosen from one of the specified groups or the substituents being chosen from two or more of the specified groups. An analogous convention applies to substituents chosen from “1 or 2” groups.

The compounds of formula (I) have activity as pharmaceuticals, in particular as modulators of chemokine receptor (especially CXCR2) activity, and may be used in the treatment (therapeutic or prophylactic) of conditions/diseases in human and non-human animals which are exacerbated or caused by excessive or unregulated production of chemokines. Examples of such conditions/diseases include (each taken independently):
(1) the respiratory tract—obstructive airways diseases including chronic obstructive pulmonary disease (COPD); asthma, such as bronchial, allergic, intrinsic, extrinsic and dust asthma, particularly chronic or inveterate asthma (e.g. late asthma and airways hyper-responsiveness); bronchitis; acute, allergic, atrophic rhinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis; seasonal rhinitis including rhinitis nervosa (hay fever) and vasomotor rhinitis; sarcoidosis, farmer's lung and related diseases, fibroid lung and idiopathic interstitial pneumonia;
(2) bone and joints—rheumatoid arthritis, seronegative spondyloarthropathies (including ankylosing spondylitis, psoriatic arthritis and Reiter's disease), Behchet's disease, Sjogren's syndrome and systemic sclerosis;
(3) skin—psoriasis, atopical dermatitis, contact dermatitis and other eczmatous dermitides, seborrhoetic dermatitis, Lichen planus, Pemphigus, bullous Pemphigus, Epidermolysis bullosa, urticaria, angiodermas, vasculitides, erythemas, cutaneous eosinophilias, uveitis, Alopecia greata and vernal conjunctivitis;
(4) gastrointestinal tract—Coeliac disease, proctitis, eosinopilic gastro-enteritis, mastocytosis, Crohn's disease, ulcerative colitis, determinate colitis, microscopic colitis, inflammatory bowel disease, irritable bowel syndrome, non-inflammatory diarrhea, food-related allergies which have effects remote from the gut, e.g., migraine, rhinitis and eczema;
(5) central and peripheral nervous system—Neurodegenerative diseases and dementia disorders, e.g. Alzheimer's disease, amyotrophic lateral sclerosis and other motor neuron diseases, Creutzfeldt-Jacob's disease and other prion diseases, HIV encephalopathy (AIDS dementia complex), Huntington's disease, frontotemporal dementia, Lewy body dementia and vascular dementia; polyneuropathies, e.g. Guillain-Barré syndrome, chronic inflammatory demyelinating polyradiculoneuropathy, multifocal motor neuropathy, plexopathies; CNS demyelination, e.g. multiple sclerosis, acute disseminated/haemorrhagic encephalomyelitis, and subacute sclerosing panencephalitis; neuromuscular disorders, e.g. myasthenia gravis and Lambert-Eaton syndrome; spinal diorders, e.g. tropical spastic paraparesis, and stiff-man syndrome: paraneoplastic syndromes, e.g. cerebellar degeneration and encephalomyelitis; CNS trauma; migraine; and stroke.
(6) other tissues and systemic disease—atherosclerosis, Acquired Immunodeficiency Syndrome (AIDS), lupus erythematosus, systemic lupus, erythematosus, Hashimoto's thyroiditis, type I diabetes, nephrotic syndrome, eosinophilia fascitis, hyper IgE syndrome, lepromatous leprosy, and idiopathic thrombocytopenia pupura; post-operative adhesions, and sepsis.
(7) allograft rejection—acute and chronic following, for example, transplantation of kidney, heart, liver, lung, bone marrow, skin and cornea; and chronic graft versus host disease;
(8) cancers—especially non-small cell lung cancer (NSCLC), malignant melanoma, prostate cancer and squamous sarcoma, and tumour metastasis, non melanoma skin cancer and chemoprevention metastases;
(9) diseases—in which angiogenesis is associated with raised CXCR2 chemokine levels (e.g. NSCLC, diabetic retinopathy);
(10) cystic fibrosis;
(11) burn wounds & chronic skin ulcers;
(12) reproductive diseases—for example disorders of ovulation, menstruation and implantation, pre-term labour, endometriosis;
(13) re-perfusion injury—in the heart, brain, peripheral limbs and other organs, inhibition of atherosclerosis.

Thus, the present invention provides a compound of formula (1), or a pharmaceutically-acceptable salt, solvate or an in vivo hydrolysable ester thereof, as hereinbefore defined for use in therapy.

Preferably the compounds of the invention are used to treat diseases in which the chemokine receptor belongs to the CXC chemokine receptor subfamily, more preferably the target chemokine receptor is the CXCR2 receptor.

Particular conditions which can be treated with the compounds of the invention are cancer, diseases in which angiogenesis is associated with raised CXCR2 chemokine levels, and inflammatory diseases such as asthma, allergic rhinitis, COPD, rheumatoid arthritis, psoriasis, inflammatory bowel diseases, osteoarthritis or osteoporosis.

As a further aspect of the present invention, the compounds of formula (1) may have utility as antagonists of the CX3CR1 receptor. Such compounds are expected to be particularly useful in the treatment of disorders within the central and peripheral nervous system and other conditions characterized by an activation of microglia and/or infiltration of leukocytes (e.g. stroke/ischemia and head trauma). In particular, the compounds are indicated for use in the treatment of neurodegenerative disorders or demyelinating disease in mammals including man More particularly, the compounds are indicated for use in the treatment of multiple sclerosis. The compounds are also indicated to be useful in the treatment of pain, rheumatoid arthritis, osteoarthritis, stroke, atherosclerosis and pulmonary arterial hypertension.

The compounds of the invention may also be used to treat diseases in which the chemokine receptor belongs to the CCR chemokine receptor subfamily, more preferably the target chemokine receptor is the CCR2b receptor.

In a further aspect, the present invention provides a compound of formula (1), or a pharmaceutically acceptable salt, solvate or in vivo hydrolysable ester thereof, as hereinbefore defined for use as a medicament.

In a still further aspect, the present invention provides the use of a compound of formula (1), or a pharmaceutically acceptable salt, solvate or in vivo hydrolysable ester thereof, as hereinbefore defined for use as a medicament for the treatment of human diseases or conditions in which modulation of chemokine receptor activity is beneficial.

In a still further aspect, the present invention provides the use of a compound of formula (1), or a pharmaceutically acceptable salt, solvate or in vivo hydrolysable ester thereof, as hereinbefore defined for use as a medicament for the treatment of asthma, allergic rhinitis, cancer, COPD, rheumatoid arthritis, psoriasis, inflammatory bowel diseases, osteoarthritis or osteoporosis.

In a further aspect, the present invention provides the use of a compound of formula (1), or a pharmaceutically acceptable salt, solvate or in vivo hydrolysable ester thereof, as hereinbefore defined in the manufacture of a medicament for use in therapy.

In a still further aspect, the present invention provides the use of a compound of formula (1), or a pharmaceutically acceptable salt, solvate or in vivo hydrolysable ester thereof, as hereinbefore defined in the manufacture of a medicament for the treatment of human diseases or conditions in which modulation of chemokine receptor activity is beneficial.

In a still further aspect, the present invention provides the use of a compound of formula (1), or a pharmaceutically acceptable salt, solvate or in vivo hydrolysable ester thereof, as hereinbefore defined in the manufacture of a medicament for the treatment of asthma, allergic rhinitis, cancer, COPD, rheumatoid arthritis, psoriasis, inflammatory bowel diseases, osteoarthritis or osteoporosis.

In the context of the present specification, the term “therapy” also includes “prophylaxis” unless there are specific indications to the contrary. The terms “therapeutic” and “therapeutically” should be construed accordingly.

The invention still further provides a method of treating a chemokine mediated disease wherein the chemokine binds to a chemokine (especially CXCR2) receptor, which comprises administering to a patient a therapeutically effective amount of a compound of formula, or a pharmaceutically acceptable salt, solvate or in vivo hydrolysable ester, as hereinbefore defined.

The invention also provides a method of treating an inflammatory disease, especially asthma, allergic rhinitis, COPD, rheumatoid arthritis, psoriasis, inflammatory bowel diseases, osteoarthritis or osteoporosis, in a patient suffering from, or at risk of, said disease, which comprises administering to the patient a therapeutically effective amount of a compound of formula (1), or a pharmaceutically acceptable salt, solvate or in vivo hydrolysable ester thereof, as hereinbefore defined.

For the above-mentioned therapeutic uses the dosage administered will, of course, vary with the compound employed, the mode of administration, the treatment desired and the disorder indicated.

The compounds of formula (1) and pharmaceutically acceptable salts, solvates or in vivo hydrolysable esters thereof may be used on their own but will generally be administered in the form of a pharmaceutical composition in which formula (I) compound/salt/solvate/ester (active ingredient) is in association with a pharmaceutically acceptable adjuvant, diluent or carrier. Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (1), or a pharmaceutically acceptable salt, solvate or in vivo hydrolysable ester thereof, as hereinbefore defined, in association with a pharmaceutically acceptable adjuvant, diluent or carrier.

The invention further provides a process for the preparation of a pharmaceutical composition of the invention which comprises mixing a compound of formula (1), or a pharmaceutically acceptable salt, solvate or in vivo hydrolysable ester thereof, as hereinbefore defined, with a pharmaceutically acceptable adjuvant, diluent or carrier. The pharmaceutical compositions may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally. Preferably the compounds of the invention are administered orally.

In addition to their use as therapeutic medicines, the compounds of formula (I) and their pharmaceutically acceptable salts, solvate or in vivo hydrolysable esters are also useful as pharmacological tools in the development and standardisation of in vitro and in vivo test systems for the evaluation of the effect of chemokine modulation activity in labatory animals such as cats, dogs, rabbits, monkeys, rats and mice, as part of the search for new therapeutic agents.

The invention further relates to combination therapies wherein a compound of formula (I) or a pharmaceutically acceptable salts, solvate or in vivo hydrolysable ester thereof, or a pharmaceutical composition or formulation comprising a compound of formula (I) is administered concurrently or sequentially with therapy and/or an agent for the treatment of any one of asthma, allergic rhinitis, cancer, COPD, rheumatoid arthritis, psoriasis, inflammatory bowel disease, irritable bowel syndrome, osteoarthritis or osteoporosis.

In particular, for the treatment of the inflammatory diseases rheumatoid arthritis, psoriasis, inflammatory bowel disease, irritable bowel syndrome, COPD, asthma and allergic rhinitis the compounds of the invention may be combined with agents such as TNF-α inhibitors such as anti-TNF monoclonal antibodies (such as Remicade, CDP-870 and D.sub2.E.sub7.) and TNF receptor immunoglobulin molecules (such as EnbreLreg.), non-selective COX-1/COX-2 inhibitors (such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin), COX-2 inhibitors (such as meloxicam, celecoxib, rofecoxib, valdecoxib and etoricoxib) low dose methotrexate, lefunomide; ciclesonide; hydroxychloroquine, d-penicillamine, auranofin or parenteral or oral gold. For inflammatory bowel disease and irritable bowel disorder further convenient agents include sulphasalazine and 5-ASAs, topical and systemic steroids, immunomodulators and immunosuppressants, antibiotics, probiotics and anti-integrins.

The present invention still further relates to the combination of a compound of the invention together with a leukotriene biosynthesis inhibitor, 5-lipoxygenase (5-LO) inhibitor or 5-lipoxygenase activating protein (FLAP) antagonist such as zileuton; ABT-761; fenleuton; tepoxalin; Abbott-79175; Abbott-85761; N-(5-substituted)-thiophene-2-alkylsulfonamides; 2,6-di-tert-butylphenol hydrazones; methoxytetrahydropyrans such as Zeneca ZD-2138; the compound SB-210661; pyridinyl-substituted 2-cyanonaphthalene compounds such as L-739,010; 2-cyanoquinoline compounds such as L-746,530; indole and quinoline compounds such as MK-591, MK-886, and BAY x 1005.

The present invention still further relates to the combination of a compound of the invention together with a receptor antagonist for leukotrienes LTB.sub4., LTC.sub4., LTD.sub4., and LTE.sub4. selected from the group consisting of the phenothiazin-3-ones such as L-651,392; amidino compounds such as CGS-25019c; benzoxalamines such as ontazolast; benzenecarboximidamides such as BUL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention together with a PDE4 inhibitor including inhibitors of the isoform PDE4D.

The present invention still further relates to the combination of a compound of the invention together with a antihistamine H.sub1. receptor antagonists such as cetirizine, loratadine, desloratadine, fexofenadine, astemizole, azelastine, and chlorpheniramine.

The present invention still further relates to the combination of a compound of the invention together with a gastroprotective H.sub2. receptor antagonist.

The present invention still further relates to the combination of a compound of the invention together with an α.sub1.- and α.sub2.-adrenoceptor agonist vasoconstrictor sympamomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride.

The present invention still further relates to the combination of a compound of the invention together with anticholinergic agents such as ipratropium bromide; tiotropium bromide; oxitropium bromide; pirenzepine; and telenzepine.

The present invention still further relates to the combination of a compound of the invention together with a β.sub1.- to β.sub4.-adrenoceptor agonists such as metaproterenol, isoproterenol, isoprenaline, albuterol, salbutamol, formoterol, salmeterol, terbutaline, orciprenaline, bitolterol mesylate, and pirbuterol; or methylxanthanines including theophylline and aminophylline; sodium cromoglycate; or muscarinic receptor (M1, M2, and M3) antagonist.

The present invention still further relates to the combination of a compound of the invention together with an insulin-like growth factor type I (IGF-1) mimetic.

The present invention still further relates to the combination of a compound of the invention together with an inhaled glucocorticoid with reduced systemic side effects, such as prednisone, prednisolone, flunisolide, triamcinolone acetonide, beclomethasone dipropionate, budesonide, fluticasone propionate, and mometasone furoate.

The present invention still further relates to the combination of a compound of the invention together with an inhibitor of matrix metalloproteases (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-12.

The present invention still further relates to the combination of a compound of the invention together with other modulators of chemokine receptor function such as CCR1, CCR2, CCR2A, CCR2B, CCR3, CCR4, CCR5, CCR6, CCR7, CCR8, CCR9, CCR10 and CCR11 (for the C-C family); CXCR1, CXCR3, CXCR4 and CXCR5 (for the C—X—C family) and CX₃CR¹ for the C—X₃—C family.

The present invention still further relates to the combination of a compound of the invention together with antiviral agents such as Viracept, AZT, aciclovir and famciclovir, and antisepsis compounds such as Valant.

The present invention still further relates to the combination of a compound of the invention together with cardiovascular agents such as calcium channel blockers, lipid lowering agents such as statins, fibrates, beta-blockers, Ace inhibitors, Angiotensin-2 receptor antagonists and platelet aggregation inhibitors.

The present invention still further relates to the combination of a compound of the invention together with CNS agents such as antidepressants (such as sertraline), anti-parkinsonian drugs (such as deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), and anti-Alzheimer's drugs such as donepezil, tacrine, COX-2 inhibitors, propentofylline or metrifonate.

The present invention still further relates to the combination of a compound of the invention together with (i) tryptase inhibitors; (ii) platelet activating factor (PAF) antagonists; (hi) interleukin converting enzyme (ICE) inhibitors; (iv) IMPDH inhibitors; (v) adhesion molecule inhibitors including VLA-4 antagonists; (vi) cathepsins; (vii) MAP kinase inhibitors; (viii) glucose-6 phosphate dehydrogenase inhibitors; (ix) kinin-B.sub1.- and B.sub2.-receptor antagonists; (x) anti-gout agents, e.g., colchicine; (xi) xanthine oxidase inhibitors, e.g., allopurinol; (xii) uricosuric agents, e.g., probenecid, sulfinpyrazone, and benzbromarone; (xiii) growth hormone secretagogues; (xiv) transforming growth factor (TGFβ); (xv) platelet-derived growth factor (PDGF); (xvi) fibroblast growth factor, e.g., basic fibroblast growth factor (bFGF); (xvii) granulocyte macrophage colony stimulating factor (GM-CSF); (xviii) capsaicin cream; (xix) Tachykinin NK.sub1. and NK.sub3. receptor antagonists selected from the group consisting of NKP-608C; SB-233412 (talnetant); and D-4418; (xx) elastase inhibitors selected from the group consisting of UT-77 and ZD-0892; (xxi) TNF α converting enzyme inhibitors (TACE); (xxii) induced nitric oxide synthase inhibitors (iNOS) or (xxiii) chemoattractant receptor-homologous molecule expressed on TH2 cells, (CRTH2 antagonists).

The compounds of the present invention may also be used in combination with osteoporosis agents such as raloxifene, droloxifene, lasofoxifene or fosomax and immunosuppressant agents such as FK-506, rapamycin, cyclosporine, azathioprine, and methotrexate;

The compounds of the invention may also be used in combination with existing therapeutic agents for the treatment of osteoarthritis. Suitable agents to be used in combination include standard non-steroidal anti-inflammatory agents (hereinafter NSATD's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, rofecoxib and etoricoxib, analgesics and intraarticular therapies such as corticosteroids and hyaluronic acids such as hyalgan and synvisc and P2X7 receptor antagonists.

The compounds of the invention can also be used in combination with existing therapeutic agents for the treatment of cancer. Suitable agents to be used in combination include:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea, gemcitabine and paclitaxel (Taxol®); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

### Pharmacological Data

### Ligand Binding Assay

[¹²⁵I]IL-8 (Thuman, recombinant) was purchased from Amersham, U.K. with a specific activity of 2,000 Ci/mmol. All other chemicals were of analytical grade. High levels of hrCXCR2 were expressed in HEK 293 cells (human embryo kidney 293 cells BCACC No. 85120602) (Lee et al (1992) J. Biol. Chem. 267 pp16283-16291). hrCXCR2 cDNA was amplified and cloned from human neutrophil mRNA The DNA was cloned into PCRScript (Stratagene) and clones were identified using DNA. The coding sequence was sub-cloned into the eukaryotic expression vector RcCMV (Invitrogen). Plasmid DNA was prepared using Quiagen Megaprep 2500 and transfected into HEK 293 cells using Lipofectamine reagent (Gibco BRL). Cells of the highest expressing clone were harvested in phosphate-buffered saline containing 0.2% (w/v) ethylenediaminetetraacetic acid (EDTA) and centrifuged (200 g, 5 min.). The cell pellet was resuspended in ice cold homogenisation buffer [10 mM HEPES (pH 7.4), 1 mM dithiothreitol, 1 mM EDTA and a panel of protease inhibitors (1 mM phenyl methyl sulphonyl fluoride, 2 μg/ml soybean trypsin inhibitor, 3 mM benzamidine, 0.5 μg/ml leupeptin and 100 μg/ml bacitracin)] and the cells left to swell for 10 minutes. The cell preparation was disrupted using a hand held glass mortar/PTFE pestle homogeniser and cell membranes harvested by centrifugation (45 minutes, 100,000 g, 4° C.). The membrane preparation was stored at −70° C. in homogenisation buffer supplemented with Tyrode's salt solution (137 mM NaCl, 2.7 mM KCl, 0.4 mM NaH₂PO₄), 0.1% (w/v) gelatin and 10% (v/v) glycerol.

All assays were performed in a 96-well MultiScreen 0.45 μm filtration plates (Millipore, U.K.). Each assay contained ˜50 pM [¹²⁵I]IL-8 and membranes (equivalent to −200,000 cells) in assay buffer [Tyrode's salt solution supplemented with 10 mM HEPES (pH 7.4), 1.8 mM CaCl₂, 1 mM MgCl₂, 0.125 mg/ml bacitracin and 0.1% (w/v) gelatin]. In addition, a compound of formula (I) according to the Examples was pre-dissolved in DMSO and added to reach a final concentration of 1% (v/v) DMSO. The assay was initiated with the addition of membranes and after 1.5 hours at room temperature the membranes were harvested by filtration using a Millipore MultiScreen vacuum manifold and washed twice with assay buffer (without bacitracin). The backing plate was removed from the MultiScreen plate assembly, the filters dried at room temperature, punched out and then counted on a Cobra γ-counter.

The compounds of formula (I) according to the Examples 1-7 were found to have pIC₅₀ values of greater than (>) 4.5.

### Intracellular Calcium Mobilisation Assay

Human neutrophils were prepared from EDTA-treated peripheral blood, as previously described (Baly et al. (1997) Methods in Enzymology 287 pp 70-72), in storage buffer [Tyrode's salt solution (137 mM NaCl, 2.7 mM KCl, 0.4 mM NaH₂PO₄) supplemented with 5.7 mM glucose and 10 mM HEPES (pH 7.4)].

The chemokine GROα (human, recombinant) was purchased from R&D Systems (Abingdon, U.K.). All other chemicals were of analytical grade. Changes in intracellular free calcium were measured fluorometrically by loading neutrophils with the calcium sensitive fluorescent dye, fluo-3, as described previously (Merritt et al. (1990) Biochem. J. 269, pp 513-519). Cells were loaded for 1 hour at 37° C. in loading buffer (storage buffer with 0.1% (w/v) gelatin) containing 5 μM fluo-3 AM ester, washed with loading buffer and then resuspended in Tyrode's salt solution supplemented with 5.7 mM glucose, 0.1% (w/v) bovine serum albumin (BSA), 1.8 mM CaCl₂ and 1 mM MgCl₂. The cells were pipetted into black walled, clear bottom, 96 well micro plates (Costar, Boston, U.S.A.) and centrifuged (200 g, 5 minutes, room temperature).

A compound of formula (I) according to the Examples was pre-dissolved in DMSO and added to a final concentration of 0.1% (v/v) DMSO. Assays were initiated by the addition of an A₅₀ concentration of GROα and the transient increase in fluo-3-fluorescence (λ_{Ex}=490 nm and λ_{Em}=520 nm) monitored using a FLIPR (Fluorometric Imaging Plate Reader, Molecular Devices, Sunnyvale, U.S.A.).

The compounds of formula (I) according to the Examples 1-4 were tested and found to be antagonists of the CXCR2 receptor in human neutrophils.

The invention will now be illustrated by the following non-limiting Examples in which, unless stated otherwise:
(i) when given Nuclear Magnetic Resonance (NMR) spectra were measured on a Varian Unity Inova 300 or 400 MHz spectrometer. ¹H NMR data is quoted in the form of delta values for major diagnostic protons, given in parts per million (ppm) relative to tetramethylsilane (TMS) as an internal standard.
(ii) Mass Spectrometry (MS) spectra were measured on a Finnigan Mat SSQ7000or Micromass Platform spectrometer.
(iii) the title and sub-titled compounds of the Examples and methods were named using the ACD/Name program (version 4.55) from Advanced Chemical Development Inc, Canada.
(iv) Normal phase column chromatography and normal phase HPLC was conducted using a silica column. Reverse phase High Pressure Liquid Chromatography (HPLC) purification was performed using either a Waters Micromass LCZ with a Waters 600 pump controller, Waters 2487 detector and Gilson FC024 fraction collector or a Waters Delta Prep 4000 or a Gilson Auto Purification System, using a Symmetry, NovaPak or Ex-Terra reverse phase silica column.
(v) The following abbreviations are used:
   AcOH acetic acid
   CHCl₃ chloroform
   DCM dichloromethane
   DMF N,N-dimethylformamide
   DMSO dimethylsulfoxide
   Et₂O diethyl ether
   EtOAc ethyl acetate
   MgSO₄ magnesium sulfate
   NMP 1-methylpyrrolidin-2-one
   THF tetrahydrofuran
   H₂O water

### EXAMPLE 1

### 3-[[2-amino-5-[[(2-fluorophenyl)methyl]thio]thiazolo[4,5-d]pyrimidin-7-yl]oxy]-propanoic acid

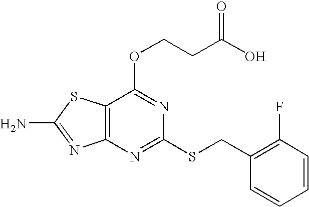

### 2-Amino-5-[[(2-fluorophenyl)methyl]thio]thiazolo[4,5-d]pyrimidin-7(4H)-one

To a solution of 3-bromo-propanoic acid (367 mg) in DMF (10 ml) was added 2-Amino-5-[[(2-fluorophenyl)methyl]thio]thiazolo[4,5-d]pyrimidin-7(4H)-one (see WO0009511) (250 mg), N,N-diisopropylethylamine (105 mg) and a catalytic amount of sodium iodide the mixture was then heated at 90° C.-100° C. for 2 days. The reaction mixture was then evaporated under reduced pressure before separating between ethyl acetate (200 ml) and water (200 ml). The aqueous phase was then re-extracted with ethyl acetate (2×200 ml). The combined organics were then dried using MgSO₄, filtered and evaporated to give a residue which was purified by reverse phase HPLC. Yield 36 mg.

MS APCI(−ve) 379[M−H]⁻

¹H NMR: (CD₃OD) δ 7.65-7.61 (1H, m), 7.33-7.29 (1H, m), 7.14-7.09 (2H, m), 4.59 (2H, s), 4.34 (2H, t), 2.70 (2H, t)

### EXAMPLE 2

### 3-[[2-amino-5-[[(2-fluorophenyl)methyl]thio]thiazolo[4,5-d]pyrimidin-7-yl]oxy]-propanamide

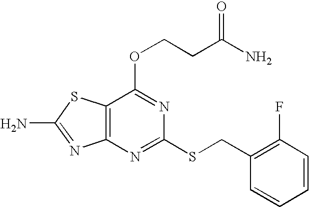

To a solution of 3-chloro-propanamide (258 mg) in DMF (10 ml) was added 2-Amino-5-[[(2-fluorophenyl)methyl]thio]thiazolo[4,5-d]pyrimidin-7(4H)-one (see WO0009511) (250 mg), N,N-diisopropylethylamine (105 mg) and a catalytic amount of sodium iodide the mixture was then heated at 90° C.-100° C. for 2 days. The reaction mixture was then evaporated under reduced pressure before separating between ethyl acetate (200 ml) and water (200 ml). The aqueous phase was then re-extracted with ethyl acetate (2×200 ml). The combined organics were then dried using MgSO₄, filtered and evaporated to give a residue which was purified by reverse phase HPLC. Yield 15 mg.

MS APCI(+ve) 378[M−H]⁻

¹H NMR: (CD₃OD) δ 7.59-7.54 (1H, m), 7.29-7.24 (1H, m), 7.13-7.05 (2H, m), 4.72 (2H, t), 4.48 (2H, s), 2.68 (2H, t)

### EXAMPLE 3

### N-[2-[[2-amino-5-[[(2-fluorophenyl)methyl]thio]thiazolo[4,5-d]pyrimidin-7-yl]oxy]ethyl]-acetamide

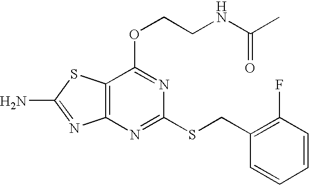

To a solution of N-(2-chloroethyl)-acetamide (292 mg) in DMF (10 ml) was added 2-Amino-5-[[(2-fluorophenyl)methyl]thio]thiazolo[4,5-d]pyrimidin-7(4H)-one (see WO0009511) (250 mg), N,N-diisopropylemylamine (105 mg) and a catalytic amount of sodium iodide the mixture was then heated at 90° C.-100° C. for 2 days. The reaction mixture was then evaporated under reduced pressure before separating between ethyl acetate (200 ml) and water (200 ml). The aqueous phase was then re-extracted with ethyl acetate (2×200 ml). The combined organics were then dried using MgSO₄, filtered and evaporated to give a residue which was purified by reverse phase HPLC. Yield 11 mg.

MS APCI(+ve) 394[M+H]⁺

¹H NMR: (CD₃OD) δ 7.57-7.53 (1H, m), 7.29-7.23 (1H, m), 7.12-7.05 (2H, m), 4.53 (2H, t), 4.47 (2H, s), 3.54 (2H, t), 1.93 (3H, s)

### EXAMPLE 4

### 1-propanol, 2-[[2-amino-5-[[(3-chloro-4-methoxyphenyl)methyl]thio]thiazolo[4,5-d]pyrimidin-7-yl]oxy]-, (2R)—

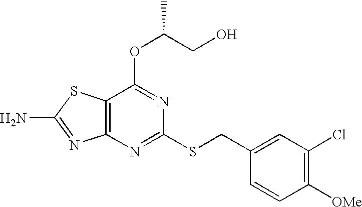

A solution of propanoic acid, 2-[[2-amino-5-[[(3-chloro-4-methoxyphenyl)methyl]thio]thiazolo[4,5-d]pyrimidin-7-yl]oxy]-, ethyl ester, (2R)-(0.23 g) in tetrahydrofuran (8 ml) at 0° C. was treated dropwise with a 2M solution of lithium borohydride in tetrahydrofuran (0.23 ml). The mixture was allowed to reach ambient temperature and further stirred for 48 h. The mixture was quenched with 2M hydrochloric acid and partitioned with ethyl acetate. The organics collected, washed with brine and then dried with MgSO4 and solvent evaporated and the crude residue purified by silica gel chromatography eluting with 5% methanol/dichloromethane and then trituration with isohexane to give the title compounds as a white solid. (71 mg)

MS APCI(+ve) 413[M+H]⁺

¹H NMR: (DMSO) δ 8.41 (s, 2H), 7.5 (s, 1H), 7.37 (d, 1H), 7.07 (d, 1H), 3.31 (m, 1H), 4.88 (t, 1H), 4.32 (s, 2H), 3.82 (s, 3H), 3.52 (s, 2H), 1.24 (d, 3H).

The propanoic acid, 2-[[2-ammo-5-[[(3-chloro-4-methoxyphenyl)methyl]thio]thiazolo[4,5-d]pyrimidin-7-yl]oxy]-, ethyl ester, (2R)— used as starting material was prepared as follows:

### i) propanoic acid, 2-[[2-amino-5-[[(3-chloro-4-methoxyphenyl)methyl]thio]thiazolo[4,5-d]pyrimidin-7-yl]oxy]-, ethyl ester, (2R)—

A solution of 2-Amino-5-[[(3-chloro-4-methoxyphenyl)methyl]thio]thiazolo[4,5-d]pyrimidin-7(4H)-one (see WO0009511) (0.71 g), triphenyl phosphine (0.53 g), (S)-ethyl lactate (0.25 ml) in tetrahydrofuran (30 ml) at 0° C. was treated dropwise with diisopropylazidodicarboxylate (0.42 ml). The mixture was allowed to reach ambient temperature and further stirred for 16 h before additional triphenyl phosphine (0.26 g) and diisopropylazidodicarboxylate (0.21 ml) was added. The mixture was further stirred for 48 h. The mixture was partitioned between ethyl acetate and water. The organics collected, washed with brine, dried (MgSO₄) and solvent evaporated. The residue purified by silica gel chromatography to give the subtitle compound as a white solid. Yield: 0.23 g

¹H NMR: (DMSO) δ 8.54 (s, 2H), 7.46 (m, 1H), 7.35 (m, 1H), 7.07 (d, 1H), 5.33 (q, 1H), 4.27 (q, 2H), 4.13 (m, 2H), 3.82 (s, 3H), 1.53 (d, 3H), 1.18 (t, 3H),

### EXAMPLE 5

### (2R)-2-({2-amino-5-[(2,3-difluorobenzyl)thio][1,3]thiazolo[4,5-d]pyrimidin-7-yl}oxy)propan-1-ol

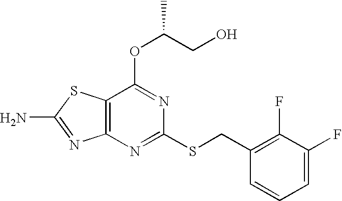

A solution of 7-((1R)-2-{[tert-butyl(dimethyl)silyl]oxy}-1-methylethoxy)-5-[(2,3-difluorobenzyl)thio][1,3]thiazolo[4,5-d]pyrimidin-2-amine (0.20 g) in methanol (3 ml) was added to a SCX cartridge. DCM followed by methanol was eluted through the cartridge, (eluent discarded). The compound was eluted with 4M ammonia in methanol/DCM (1:9), the relevant fractions were concentrated in vacuo to give a white solid which was washed with iso-hexane, ether and DCM. The compound was purified by column chromatography on silica gel using methanol/DCM (1:9) as eluent, to give the title compound as a white solid. Yield: 28 mg

MS APCI(+ve) 385[M+H]⁺

¹H NMR: (DMSO) δ 8.48 (s, 2H), 7.43-7.27 (m, 2H), 7.19-7.10 (m, 1H), 5.32-5.23 (m, 1H), 4.88 (t, 1H), 4.45 (dd, 2H), 3.52 (t, 2H), 1.21 (d, 3H)

The 7-((1R)-2-{[tert-butyl(dimethyl)silyl]oxy}-1-methylethoxy)-5-[(2,3-difluorobenzyl)thio][1,3]thiazolo[4,5-d]pyrimidin-2-amine used as starting material was prepared as follows:

### i)-7-((1R)-2-{[tert-butyl(dimethyl)silyl]oxy}-1-methylethoxy)-5-[(2,3-difluorobenzyl)thio][1,3]thiazolo[4,5-d]pyrimidin-2-amine

The subtitle compound was prepared according to the procedure outlined in example 4 step i) using 2-Ammo-5-(2,3-difluoro-benzylsulfanyl)-(0.51 g), 1-(tert-Butyl-dimethyl-silanyloxy)-propan-2-ol-(S) (0.47 g), triphenyl phosphine (0.73 g), tetrahydrofuran (30 ml) and diisopropylazidodicarboxylate (0.55 ml), to give the subtitle compound as a white solid. Yield: 0.20 g

MS APCI(+ve) 499[M+H]⁺

### EXAMPLE 6

### (2S)-2-({2-amino-5-[(2,3-difluorobenzyl)thio][1,3]thiazolo[4,5-d]pyrimidin-7-yl}oxy)propan-1-ol

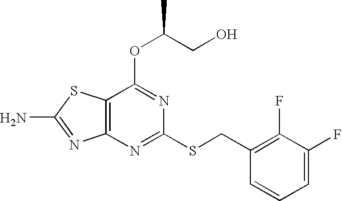

To a solution of 7-((1S)-2-{[tert-butyl(dimethyl)silyl]oxy}-1-methylethoxy)-5-[(2,3-difluorobenzyl)thio][1,3]thiazolo[4,5-d]pyrimidin-2-amine (40 mg) in THF (5 ml) was added TBAF (0.16 ml). The mixture was stirred at ambient temperature for 3 h The mixture was partitioned between ethyl acetate and water. The organics collected, washed with brine, dried (MgSCU) and solvent evaporated. The residue was purified by silica gel chromatography using methanol/DCM (1:9) as eluent, then further purified by reverse phase HPLC (symmetry as the stationary phase and ammonium acetate/acetonitrile as the mobile phase) The mixture was then titurated with methanol and DCM to give the title compound as a white solid. Yield: 10 mg

MS APCI(+ve) 385[M+H]⁺

¹H NMR: (DMSO) δ8.48 (s, 2H), 7.42-7.28 (m, 2H), 7.19-7.11 (m, 1H), 5.32-5.22 (m, 1H), 4.45 (dd, 2H), 3.56-3.48 (m, 2H), 1.21 (d, 3H)

The 7-((1S)-2-{[tert-butyl(dimethyl)silyl]oxy}-1-methylethoxy)-5-[(2,3-difluorobenzyl)thio][1,3]thiazolo[4,5-d]pyrimidin-2-amine used as starting material was prepared as follows:

### i) 7-((1S)-2-{[tert-butyl(dimethyl)silyl]oxy}-1-methylethoxy)-5-[(2,3-difluorobenzyl)thio][1,3]thiazolo[4,5-d]pyrimidin-2-amine

The subtitle compound was prepared according to the procedure outlined in example 4 step i) using 2-Amino-5-(2,3-difluoro-benzylsulfanyl)-6H-thiazolo[4,5-d]pyrimidin-7-one (0.51 g), 1-(tert-Butyl-dimethyl-silanyloxy)-propan-2-ol-(R) (0.47 g), triphenyl phosphine (0.73 g), tetrahydrofuran (30 ml) and diisopropylazidodicarboxylate (0.55 ml), to give the subtitle compound as a white solid. Yield: 40 mg

MS APCI(−ve) 497[M−H]⁻

### EXAMPLE 7

### 5-[(2,3-difluorobenzyl)thio]-7-[(1R)-2-hydroxy-1-methylethoxy][1,3]thiazolo[4,5-d]pyrimidin-2(3H)-one

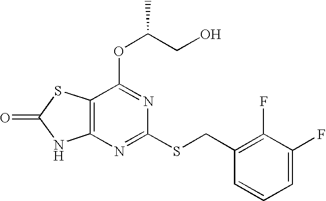

A solution of lithium borohydride (165 μL) in THF (2.0 M) was added dropwise to a stirred solution of ethyl (2R)-2-({5-[(2,3-difluorobenzyl)thio]-2-oxo-2,3-dihydro[1,3]thiazolo[4,5-d]pyrimidin-7-yl}oxy)propanoate (the product of step vi) (70 mg) in THF (10 mL) at 5° C. The mixture was allowed to warm to room temperature and stirred for a further 18 h. The reaction mixture was quenched with saturated aqueous NH₄Cl solution (10 mL) and extracted with EtOAc (3×10 mL). Combined organic extracts were washed with a saturated solution of brine (10 mL), dried (MgSO₄) and evaporated to dryness in vacuo. This crude residue was purified by reverse phase HPLC using a gradient of 75:25 to 5:95 mixture of 0.2% aqueous ammonium acetate solution and acetonitrile as eluent to give the title compound as a white solid (22 mg).

MS APCI(−ve) 384[M−H]⁻

¹H NMR: δ (DMSO-d₆) 1.21 (3H, d), 3.52 (2H, d), 4.46 (2H, dd), 4.91 (1H, s), 5.29 (1H, q), 7.17 (1H, t), 7.45-7.29 (2H, m).

The intermediates for this compound were prepared as follows:

### i) 6-amino-2-[[(2,3-difluorophenyl)methyl]thio]-4-pyrimidionol

Aqueous sodium hydroxide solution (46-48% w/w; 24 mL) followed by H₂O (40 mL) was added to a stirred suspension of 4-ammo-6-hydroxy-2-mercaptopyrimidine monohydrate (67.7 g) in a mixture of water (920 mL) and THF (300 mL). The resulting hazy, pale yellow solution was cooled to 20° C. before adding 2,3-difluorobenzyl bromide (83.0 g) uniformly over 25 mins, to yield a white precipitate. The mixture was stirred at ambient temperature for 3.5 h, the product collected and washed twice with a mixture of H₂O (68 mL) and THF (24 mL), to afford the subtitle compound as a white solid (101.89 g). ¹H NMR: δ (DMSO-d₆) 4.39 (2H, s), 5.01 (1H, s), 6.58 (2H, br.s), 7.15 (1H, m), 7.34 (1H, m), 7.44 (1H, t), 11.45 (1H, br.s).

### ii) 7-ammo-5-[[(2,3-difluorophenyl)methyl]thio][1,3]oxathiolo[5,4-d]pyrimidin-2-one

Chlorocarbonylsulfenyl chloride (4.89 g) was added over 7 mins, followed by THF (2 mL), to a stirred suspension of 6-amino-2-[[(2,3-difluorophenyl)methyl]thio]-4-pyrimidinol (the product of step i) (9.58 g) in THF (96 mL). The reaction mixture was stirred for 40 mins and the resulting precipitate collected by filtration, washing twice with THF (19 mL), to afford the subtitle compound as a pale yellow solid (11.31 g). ¹H NMR: δ (DMSO-d6) 4.39 (2H, s), 5.82 (1H, br.s), 7.16 (1H, m), 7.34 (1H, m), 7.45 (1H, t), 7.89 (1H.br.s),

### iii) 7-chloro-5-[[(2,3-difluorophenyl)methyl]thio]thiazolo[4,5-d]pyrimidin-2-(3H)-one

N,N-diethylaniline (2.46 g), followed by acetonitrile (5 mL), and then phosphorus oxychloride (7.41 g), followed by acetonitrile (5 mL) was first added to a stirred suspension of 7-amino-5-[[(2,3-difluorophenyl)methyl]thio][1,3]oxathiolo[5,4-d]pyrimidin-2-one (the product of step ii) (5.03 g) and benzyltrimethylammonium chloride (2.58 g) in acetonitrile (25 mL) at 50° C. The reaction mixture was heated to reflux and maintained at this temperature for 36 h, before cooling to ambient temperature and adding to H₂O (25 mL) at 50° C. with stirring over 30 mins. An additional acetonitrile (5 ml) rinse of the reaction vessel was added to the drown-out mixture, before heating to 75° C. and slowly cooling to 25° C. at <0.5° C./min. The resulting mixture was held at 25° C. for 30 mins and then collected by filtration, washing four times with water (25 mL), to afford the subtitle compound as an off-white solid (3.5 g).

¹H NMR: δ (DMSO-d₆) 7.45 (1H, t), 7.38 (1H, m), 7.22 (1H, m), 4.50 (2H, s), 3.43 (1H, br.s).

### iv) 7-chloro-5-[(2,3-difluorobenzyl)thio]-3-(tetrahydro-2H-pyran-2-yl)[1,3]thiazolo[4,5-d]pyrimidin-2(3H)-one

Para-toluenesulfonic acid (10 mg), followed by 3,4-dihydro-2H-pyran (0.56 mL) was added to a solution of 7-chloro-5-[[(2,3-difluorophenyl)methyl]thio]thiazolo[4,5-d]pyrimidin-2-(3H)-one (the product of step iii) (1.67 g) in toluene (15 mL) and the mixture heated at 60° C. and stirred at that temperature for 3 h The mixture was allowed to cool to room temperature, and toluene (10 mL) and saturated aqueous sodium bicarbonate solution (30 mL) were added and then further stirred for 1 h. The layers were separated and the organic layer was washed with saturated brine solution (10 mL), dried (MgSO₄) and evaporated to dryness in vacuo. The resulting crude oil was dissolved into Et₂O (20 mL) and MeOH (few drops) and evaporated again to give a ‘wet’ solid. This material was triturated with Et₂O and filtered to give the subtitle compound as a yellow solid (1.86 g). ¹H NMR: δ (DMSO-d₆) 1.50 (2H, m), 1.70 (2H, m), 1.90 (1H, m), 2.60 (1H, m), 3.60 (1H, m), 3.99 (1H, d), 4.54 (2H, s), 5.59 (1H, m), 7.20-7.13 (1H, m), 7.44-7.31 (2H, m).

### v) ethyl (2R)-2-{[5-[(2,3-difluorobenzyl)thio]-2-oxo-3-(tetrahydro-2H-pyran-2-yl)-2,3-dihydro[1,3]thiazolo[4,5-d]pyridimin-7-yl]oxy}propanoate

Ethyl lactate (0.28 mL), followed by sodium hydride (80 mg) was added to a solution of 7-chloro-5-[(2,3-difluorobenzyl)thio]-3-(tetrahydro-2 if-pyran-2-yl)[1,3]thiazolo[4,5-f]pyrimidin-2(3H)-one (the product of step iv) (0.43 g) in THF (20 mL). The resulting mixture was stirred under nitrogen at room temperature for 18 h before quenching with saturated aqueous ammonium chloride solution (20 mL). This was then extracted with EtOAc (3×20 mL) and the combined organic extracts were washed with saturated brine solution (20 mL), dried (MgSO₄) and evaporated to dryness in vacuo to give the crude subtitle compound as a yellow oil (680 mg).

¹H NMR: δ (DMSO-d6) 1.56-1.11 (7H, m), 1.74-1.57 (2H, m), 1.95-1.86 (1H, m), 2.74-2.59 (1H, m), 3.64-3.55 (1H, m), 4.05-3.97 (1H, m), 4.18-4.07 (3H, m), 4.55-4.41 (2H, m), 5.47-5.37 (1H, m), 5.59 (1H, dt), 7.17 (1H, dd), 7.44-7.28 (2H, m),

### vi) ethyl(2R)-2-({5-[(2,3-difluorobenzyl)thio]-2-oxo-2,3-dihydro[1,3]thiazolo[4,5-d]pyrimidin-7-yl}oxy)propanoate

A solution of ethyl (2R)-2-{[5-[(2,3-difluorobenzyl)thio]-2-oxo-3-(tetrahydro-2H-pyran-2-yl)-2,3-dihydro[1,3]thiazolo[4,5-d]pyrimidin-7-yl]oxy}propanoate (the product of step v) (511 mg) in a mixture of acetonitrile (20 mL), water (3.5 mL), THF (3.0 mL) and 1N aqueous HCl (2.0 mL) was heated at 60° C. for 1.5 h The mixture was allowed to cool to room temperature, was diluted with H₂O (20 mL) and then extracted with EtOAc (3×20 mL). Combined organic extracts were washed with saturated brine solution (20 mL), dried (MgSO₄) and evaporated to dryness in vacuo. The resulting crude residue was purified by flash column chromatography on silica gel, eluting with EtOAc/isohexane (1:9 to 3:7 gradient) as eluent to afford the subtitle compound as a an oil (220 mg).

MS APCI(+ve) 428[M+H]⁺

¹H NMR: δ (DMSO-d6) 1.13 (3H, t), 1.53 (3H, d), 4.11 (2H, ddd), 4.42 (2H, dd), 5.40 (1H, q), 7.21-7.13 (1H, m), 7.42-7.27 (2H, m), 13.10 (1H, s),

1. A compound of general formula (I) 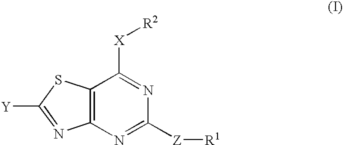
wherein
R¹ is a group selected from C₃₋₇-carbocyclyl, C₁₋₈alkyl, C₂₋₆alkenyl and C₂₋₆alkynyl; wherein the group is optionally substituted by 1, 2 or 3 substituents independently selected from fluoro, nitrile, —OR⁴, —NR⁵R⁶, —CONR⁵R⁶, —COOR⁷, —NR⁸COR⁹, —SR¹⁰, SO₂NR⁵R⁶, —NR⁸SO₂R⁹, phenyl or heteroaryl; wherein phenyl and heteroaryl are optionally substituted by 1, 2 or 3 substituents independently selected from halo, cyano, nitro, —OR⁴, —NR⁵R⁶, —CONR⁵R⁶, —COOR⁷, —NR⁸COR⁹, —SR¹⁰, —SO₂R¹⁰, —SO₂NR⁵R⁶, —NR⁸SO₂R⁹, C₁₋₆alkyl and trifluoromethyl;
X is —CH₂—, a bond, oxygen, sulphur, sulphoxide, or sulphone;
Z is —CH₂—, a bond, oxygen, sulphur, sulphoxide, sulphone or —NR⁵;
R² is C₃₋₇carbocyclyl, optionally substituted by 1, 2 or 3 substituents independently selected from: fluoro, —OR⁴, NR⁵R⁶—CONR⁵R⁶, —COOR⁷, —NR⁸COR⁹, —SR¹⁰, —SO₂R¹⁰, —SO₂NR⁵R⁶, —NR⁸SO₂R⁹;
or R² is a 3-8 membered ring optionally containing 1, 2 or 3 atoms selected from O, S, —NR⁸ and whereby the ring is optionally substituted by 1.2 or 3 substituents independently selected from C₁₋₃alkyl, fluoro, —OR⁴, —NR⁵R⁶—CONR⁵R⁶, —COOR⁷, —NR⁸COR⁹, —SR¹⁰, —SO₂R¹⁰, —SO₂NR⁵R⁶, —NR⁸SO₂R⁹;
or R² is phenyl or heteroaryl, each of which is optionally substituted by 1, 2 or 3 substituents independently selected from halo, cyano, nitro, —OR⁴, NR⁵R⁶, —CONR⁵R⁶, —NR⁸COR⁹, —SO₂NR⁵R⁶, —NR⁸SO₂R⁹, C₁₋₆alkyl and trifluoromethyl;
or R² is a group selected from C₁₋₈alkyl, C₂₋₆alkenyl or C₂₋₆alkynyl wherein the group is substituted by 1, 2 or 3 substituents independently selected from hydroxy, amino, C₁₋₆alkoxy, C₁₋₆alkylamino, di(C₁₋₆alkyl)amino, N(C₁₋₆alkyl)-N-(phenyl)amino, N—C₁₋₆alkylcarbamoyl, N,N-di(C₁₋₆alkyl)carbamoyl, N-(C₁₋₆alkyl)-N-(phenyl)carbamoyl, carboxy, phenoxyoarbonyl, —NR⁸COR⁹, —SO₂R¹⁰, —SO₂NR⁵R⁶, —NR⁸SO₂R⁹— and —CONR⁵R⁶;
Y is selected from hydrogen, hydroxyl, halo, NR³R⁴, and —NR⁸SO₂R⁹;
R³ and R⁴ each independently represent a hydrogen atom, or a 4-piperidinyl group, or R³ and R⁴ each independently represent a C₃₋₆ cycloalkyl or C₁-C₈ alkyl group, which groups may be optionally substituted by 1, 2 or 3 substituent groups independently —SO₂R¹⁰, —SO₂NR⁵R⁶, —NR⁸SO₂R⁹, morpholinyl, C₁-C₄ alkyl, C₃-C₆ cycloalkyl, tetrahydrofuranyl and aryl, wherein an aryl group may be optionally substituted by 1, 2 or 3 substituents independently selected from halo, cyano, nitro, —NR⁵R⁶, —CONR⁵R⁶, —OR⁷, —NR⁸COR⁹, —SO₂NR⁵R⁶, —NR⁸SO₂R⁹, C₁-C₆ alkyl and trifluoromethyl,
or R³ and R⁴ together with the nitrogen atom to which they are attached form a 4- to 7-membered saturated heterocyclic ring system, which ring system may be optionally substituted by one or more substituent groups independently selected from 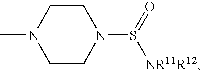
—NR⁵R⁶, —CONR⁵R⁶, —OR⁷, —COOR¹⁰, —NR⁸COR⁹, and C₁-C₆ alkyl optionally substituted by 1, 2 or 3 substituents independently selected from halogen atoms and —NR¹¹R¹² and —OR⁷ groups;
R⁵ and R⁶ are independently hydrogen or a group selected from C₁₋₆alkyl and phenyl wherein the group is optionally substituted by 1, 2 or 3 substituents independently selected from halo, phenyl, —OR¹⁴, —NR¹⁵R¹⁶, —COOR¹⁴, —CONR¹⁵R¹⁶, —NR¹⁵COR¹⁶, —SO₂R¹⁰, —SONR¹⁵R¹⁶ and NR¹⁵SO₂R¹⁶
R⁷ and R⁹ each independently represent a hydrogen atom or a C₁-C₆, particularly C₁-C₄, alkyl (e.g. methyl, ethyl, propyl, butyl, pentyl or hexyl) or phenyl group, each of which may be optionally substituted by one or more (e.g. one, two, three or four) substituent groups independently selected from halogen atoms (e.g. fluorine, chlorine, bromine or iodine), phenyl, —OR¹⁷ and —NR¹⁵R¹⁶; and
each of R⁸, R¹⁰, R¹¹, R¹², R¹⁵, R¹⁶ and R¹⁷ independently represents a hydrogen atom or a C₁-C₆, particularly C₁-C₄, alkyl (e.g. methyl, ethyl, propyl, butyl, pentyl or hexyl) or phenyl group
or a pharmaceutically acceptable salt or solvate thereof.

2. A compound or a pharmaceutically acceptable salt or solvate thereof as claimed in claim 1 wherein R¹ is a C₁₋₄ alkyl group optionally substituted by a phenyl or heteroaryl group optionally substituted by 1, 2 or 3 substituents independently selected from fluoro, chloro, bromo, methoxy, methyl and trifluoromethyl.

3. A compound or a pharmaceutically acceptable salt or solvate thereof as claimed in claim 1 wherein X is a bond, —CH₂—, oxygen, or sulphur.

4. A compound or a pharmaceutically acceptable salt or solvate thereof as claimed in claim 1 wherein R² is C₁₋₄ substituted with 1 or 2 hydroxy groups, carboxy, —NHCOC₁₋₄alkyl or —CONR⁵R⁶ wherein R⁵ and R⁶ are either hydrogen or C₁₋₄alkyl.

5. A compound, or a pharmaceutically acceptable salt or solvate thereof as claimed in claim 1 wherein Y is hydroxyl, —NR³R⁴ or —NR⁸SO₂R⁹ wherein R³, R⁴, R⁸ are either hydrogen or C₁₋₄alkyl and R⁹ is either C₁₋₄alkyl or trifloromethyl.

6. A compound or a pharmaceutically acceptable salt or solvate thereof as claimed in claim 1 wherein Z is a bond or sulphur.

7. A compound selected from the group consisting of:
3-[[2-amino-5-[[(2-fluorophenyl)methyl]thio]thiazolo[4,5-d]pyrimidin-7-yl]oxy]-propanoic acid,
3-[[2-amino-5[[(2-fluorophenyl)methyl]thio]thiazolo[4,5-d]pyrimidin-7-yl]oxy]-propanamide, N-[2-[[2-amino-5-[[(2-fluorophenyl)methyl]thio]thiazolo[4,5-d]pyrimidin-7-yl]oxy]ethyl]-acetamide,
1-propanol, 2-[[2-amino-5-[[(3-chloro-4-methoxyphenyl)methyl]thio]thiazolo[4,5-d]pyrimidin-7-yl]oxy]-, (2R)—,
(2R)-2-({2-amino-5-[(2,3-difluorobenzyl)thio][1,3]thiazolo[4,5-d]pyrimidin-7-yl}oxy)propan-1-ol, and
(2S)-2({2-amino-5-[(2,3-difluorobenzyl)thio][1,3]thiazolo[4,5-d]pyrimidin-7-yl}oxy)propan-1-ol
5[(2,3-difluorobenzyl)thio]-7-[(1R)-2-hydroxy-1-methylethoxy][1,3]thiazolo[4,5-d]pyrimidin-2(3H)-one
or a pharmaceutically acceptable salt or solvate thereof.

8. (canceled)

9. A method of treating asthma, allergic rhinitis, COPD, inflammatory bowel disease, osteoarthritis, osteoporosis, rheumatoid arthritis, or psoriasis, the method comprising administering to a subject a compound of claim 1, or a pharmaceutically acceptable salt, or solvate thereof.

10. A method of treating cancer, the method comprising administering to a subject a compound of claim 1, or a pharmaceutically acceptable salt, or solvate thereof.

11. A method of treating a human disease or condition in which modulation of chemokine receptor activity is beneficial, the method comprising administering to a human subject a compound as claimed in claim 1, or a pharmaceutically acceptable salt, or solvate thereof.

12-13. (canceled)

14. A pharmaceutical composition comprising a compound, or a pharmaceutically acceptable salt, or solvate thereof according to claim 1; and a pharmaceutically-acceptable diluent or carrier.

15. A process for the preparation of a compound of formula (I) according to claim 1 or a pharmaceutically acceptable salt or solvate thereof, which comprises the steps of:
(i) reacting a compound of general formula (III); wherein L is a leaving group and PG is a suitable protecting group 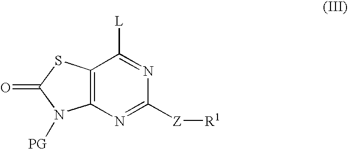
with a suitable nucleophile in the presence or absence of a suitable base and solvent
(ii) when X represents —O— or —S—, and R¹, Z and Y are as defined in formula (I), with the proviso that Y is not hydroxyl, reacting a compound of general formula (II) 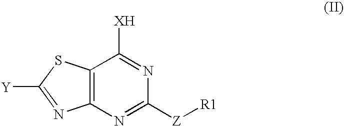
with a suitable alkylhalide (R²-L) wherein R² is as defined in formula (I) and L is a leaving group under Mitsunobu reaction conditions using a trialkyl- or triaryl-phosphine and dialkylazidodiearboxylate in the presence of a suitable base and solvent; or
(iii) for compounds of formula (I), wherein X and/or Z are sulphoxide or sulphone and R¹ and R² are as defined hereinbefore, by further reaction of compounds of formula (I), wherein X and/or Z are sulphur, with a suitable oxidising reagent;
and optionally thereafter, one or more of steps (i), (ii), (iii), (iv), or (v) in any order;
i) removing any protecting groups;
ii) converting the compound of formula (I) into a further compound of formula (I)
iii) forming a salt.

16. A combination therapy which comprises administering a compound of formula (I) or a pharmaceutically acceptable salt, or solvate thereof, or a pharmaceutical composition or formulation comprising a compound of formula (I), concurrently or sequentially with other therapy and/or another pharmaceutical agent.

17. A combination therapy as claimed in claim 16 for the treatment of asthma, allergic rhinitis, COPD, inflammatory bowel disease, irritable bowel syndrome, osteoarthritis, osteoporosis, rheumatoid arthritis, or psoriasis.

18. A combination therapy as claimed in claim 16 for the treatment of cancer.

19. A pharmaceutical composition which comprises a compound of formula (1) or a pharmaceutically acceptable salt, or solvate thereof, in conjunction with another pharmaceutical agent.

20. A pharmaceutical composition as claimed in claim 19 for the treatment of asthma, allergic rhinitis, COPD, inflammatory bowel disease, irritable bowel syndrome, osteoarthritis, osteoporosis, rheumatoid arthritis, or psoriasis.

21. A method of treating cancer, the method comprising administering to a subject a pharmaceutical composition as claimed in claim 19.
